(19) United States
(12) Patent Application Publication
(10) Pub. No.: US 2025/0082474 A1
(43) Pub. Date: Mar. 13, 2025

(54) SHAPED TISSUE GRAFT AND PROCESS TO MAINTAIN PROPERTIES

(71) Applicant: Restoration Biologics LLC, Flagstaff, AZ (US)

(72) Inventors: Suzanne M. TABBAA, Flagstaff, AZ (US); William D. BUGBEE, San Diego, CA (US); Farshid GUILAK, Clayton, MO (US)

(21) Appl. No.: 18/729,386

(22) PCT Filed: Jan. 19, 2023
(86) PCT No.: PCT/US2023/011154

§ 371 Date: Jul. 16, 2024

Related U.S. Application Data
(60) Provisional application No. 63/300,830, filed on Jan. 19, 2022.

(51) Int. Cl. A61F 2/30; A61F 2/28; A61F 2/46

(52) U.S. Cl. CPC A61F 2/30942; A61F 2/4644; A61F 2002/2835

(57) ABSTRACT

A patient-specific tissue graft and a method of forming the graft is disclosed. A 3D image of a patient's targeted implant site and surrounding anatomy is obtained to note the boundary of the implant site. A 3D model of the graft is formed by utilizing the boundary of the implant site, a surface curvature, extrapolated from the patient's target implant site surrounding anatomy, and a depth, defined by a plane parallel to a plane normal to a point of curvature at the surface. Donor tissue is chosen that has a location with a close approximation to the 3D model of the graft. The donor tissue is visualized to determine a cutting path to shape or modify the architecture of the tissue. A cutting path is determined with imaging technology and computational methods. The graft is cut out of the donor tissue utilizing a fluid cutting process.

### CROSS REFERENCE TO RELATED APPLICATIONS

This application claims the benefit of U.S. Provisional Application No. 63/300,830 titled “Patient Specific Tissue Graft and Process to Maintain Properties” and filed on Jan. 19, 2022, which is incorporated herein by reference.

### FIELD OF THE INVENTION

The invention pertains to the art of tissue grafts. More specifically, the invention describes a cutting, shaping, and tissue modification technique using a water-cutter to minimize tissue damage and improve native viability, matrix integrity, and size matching of the tissue graft.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Tissue grafts include hard or soft tissues from various sources including human or animals (e.g., allograft, xenograft, autograft) are used as an implantable to repair tissue defects. Tissue grafts are processed using various physical techniques to cut, shape, separate layers, or modify the architecture of the graft to generate a specific form for a clinical application. For Example, U.S. Pat. No. 10,251,751 B2 describes traditional methods to cut grooves in osteochondral grafts. The device in this patent also requires grooves to be cut completely through the bone and allow bending only at the cartilage layer. U.S. Pat. No. 10,722,370B2 also describes traditional methods to cut grooves in osteochondral grafts. The device in this patent also requires grooves to be cut completely through the bone and allow bending only at the cartilage layer. These techniques can utilize lasers, drills, saws, blades, mechanical cutters, or other methods to shape or modify a tissue. Such techniques are generally low through-put and requirement significant user oversight, involvement, and monitoring. Furthermore, lasers and mechanical approaches can generate heat and lead to tissue damage, adversely affect tissue architecture, and cause cell death of the graft and adjacent tissues. Additionally, mechanical and laser approaches limit the ability to generate patient-specific customizable tissues. What is needed in the art is a system and method that maintains the native matrix at the surface after cutting and can have a variety of cutting configurations.

### SUMMARY OF THE INVENTION

This invention describes a novel tissue graft of various forms with precise architecture and maintenance of the tissue viability and native micro-structure of the tissue. The tissue grafts are shaped and modified using a water-cutter technique to minimize tissue damage and improve native viability and matrix integrity of the tissue. A water-cutter involves the use of a high-pressure/high-velocity water jet, with or without a particulate abrasive additive (degradable or non-degradable) as an enhancer. The invention can involve the use of biocompatible, non-degradable, degradable, and/or particulates to enhance the water-cutter technique. This invention also describes the ability to generate 3D patient-specific tissue shapes and architectures using a computer numerical control (CNC) water-cutter technique or a water-cutter system that is controlled by a pre-programmed computer software that utilizes patient specific inputs such as imaging to generate custom patient-specific tissues. The donor tissue can be matched to the patient specific imaging by using laser or light scans and software that can image and create 3D models of the contour, architecture, and shape of the tissue The CNC water cutter system can control the robotic arm to create complex 3D tissue architectures and shapes. Direct imaging of the surface roughness or topography can be used during cutting or treatment as feedback to inform the process.

### BRIEF DESCRIPTION OF THE FIGURES

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a schematic view of a water cutter for cutting tissue grafts.

FIG. 2 shows schematic views a whole osteochondral tissue and pre-cut osteochondral cores and osteochondral tissue shapes formed by water cutting.

FIG. 3 processing steps to generate patient-specific tissue grafts shown in FIG. 2.

FIG. 4 shows a tissue graft cut with water-cutting maintains native matrix and micro-architecture at the surface.

FIG. 5 shows a tissue graft cut with traditional power technique adulterates the surface, causing compaction of the bone and reduction in porosity, which can reduce nutrient transport, cell viability, and tissue integration upon implantation of the graft.

FIG. 6 shows cartilage tissue adjacent to cutting with waterjet contains more viable cells than tradition cutting.

FIG. 7 shows cartilage tissue adjacent to cutting with traditional cutting with increased levels of dead cells. Cell viability has been shown to be an important factor in graft integration and longevity in vivo.

### DETAILED DESCRIPTION OF THE PREFERRED EMBODIMENTS

### Tissue Shaping, Cutting, and Modifications Using a Water Cutter System

Embodiments of aspects of the present invention relate to providing an enhanced tissue graft by providing a process to size and shape the tissue into various forms or architectures while reducing the amount of damage to the viability or tissue architecture. Tissue grafts are sourced from any tissues derived from human or animals (e.g., allograft, xenograft, autograft). Tissues can include any tissue type include but not limited to musculoskeletal, neural, dermal, cardiovascular, ocular, nasal, costal, adipose, or any tissue or organ type of the body, etc. Tissues are typically shaped, sliced, and modified using various mechanical approaches.

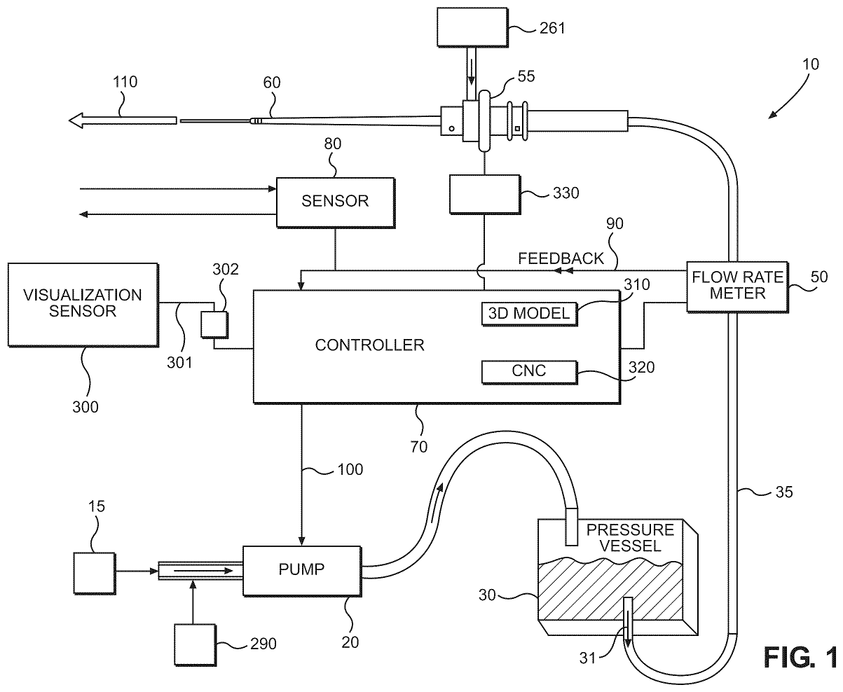

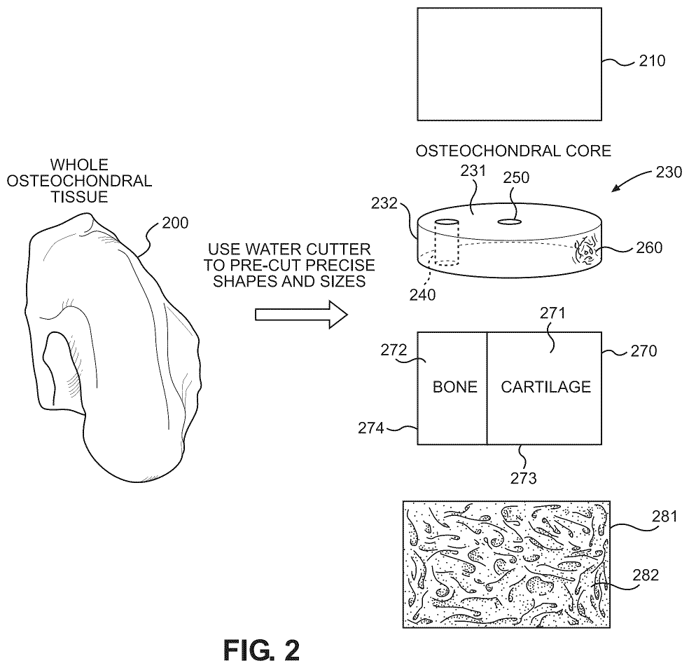

This invention describes the shaping and modification of tissues using a water cutter system. Referring to FIG. 1, there is shown a water cutter system 10. Fluid from a source 15 enters a pump 20 and is forwarded to a pressure vessel 30 via a tube 31. An output 32 of the pressure vessel 30 is forwarded along a tube 35 to a flow rate meter 50. An output 51 of flow rate meter 50 travels to a manifold 55 and then to a nozzle 60 with a depth cut sensor 70. The sensor may be, for example, an ultrasonic sensor or an optical sensor for verifying the cut depth. A signal may be sent from the sensor 70 and reflected off of the cutting region of the bone such that real-time cutting depth information can be transmitted to controller 80 to verify accuracy and/or adjust the fluid flow, if necessary.

An adjustment of the diameter of the nozzle 60 is controlled by a controller 80 employing a feedback loop 90, 100 including the controller 70, the cut depth sensor 70 and the pump 20. Controller 80 may be a computer, a central processing unit, a microcontroller, ASIC, or other control circuitry. Fluid flow at flow rate meter 50 is controlled throughout the cut of a particular section of bone. More particularly, cutting area, shape of a jet 110, and pressure, for example, of the cutting fluid can be precisely controlled via the continuous feedback loop.

Advantageously, cutting pressure can be controlled via the feedback loop 90, 100 and the adjustable nozzle 60 such that flow rate meter 50 outputs a minimum pressure sufficient to cut a particular section of bone in order to minimize damage to the surrounding soft tissue. As bone is heterogeneous, the flow rate can be continuously adjusted to maintain minimum sufficient pressure throughout the depth of the cut as different density of bone is encountered.

The water cutter system 10 preferably used combination with a particulate abrasive additive (degradable or non-degradable) as an enhancer. The additive is preferably added to from a source 261 to the manifold 55 so that the additive is incorporated into the jet 110. The additive involves biocompatible and biodegradable materials which could include but is not limited to salt crystals, biodegradable polymers, and other biomaterials. The particulate biomaterial properties (size, chemistry, architecture) used for the abrasive is tailored to various applications. The particulate is used to enhance the cutting efficiency and to modify the tissue roughness and micro-architecture. In another embodiment, the water cutter system uses physiological buffered solutions such as phosphate buffered saline to maintain viability and biological properties of the tissue. Such solutions are preferably added from a source 290 before the pump 20 but could be added in other places. The source 290 is preferably employed to add numerous different components to the water supplied at water source 15.

The water cutter system 10 is preferably employed in conjunction with an automated visualization sensor or system 300 to separate tissue types of varying composition (e.g. dermal tissue, amnion, cartilage from calcified cartilage layer, bone, etc.). This system is connected to controller 70 by communication line 301 and sends data 302 back to controller 70 which may be converted to a 3D model 310. Controller 70 is programmed to use various techniques such as machine vision, object recognition, image segmentation, convolutional neural networks, artificial intelligence, etc. to assist in cutting and shaping of distinct tissue shapes and to separate or cut specific tissues within complex composite tissues such as an osteochondral graft. The visualization system 300 is preferably employed to initiate or adjust a CNC program 320.

### Patient-Specific Tissue Grafts and Processing Methods

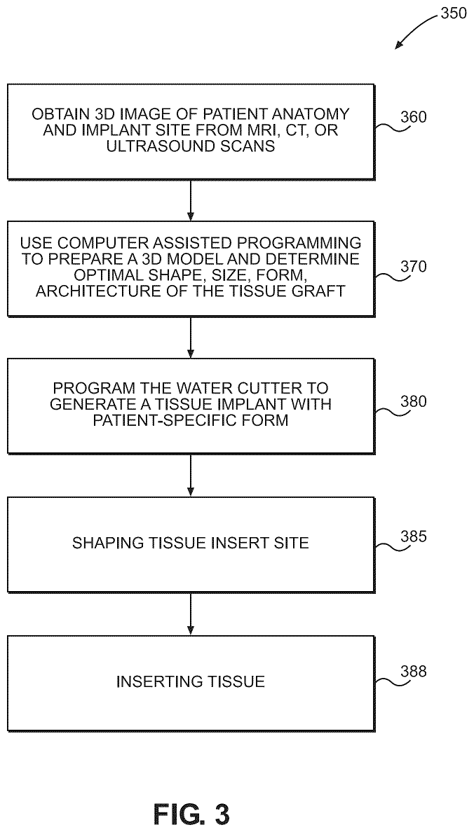

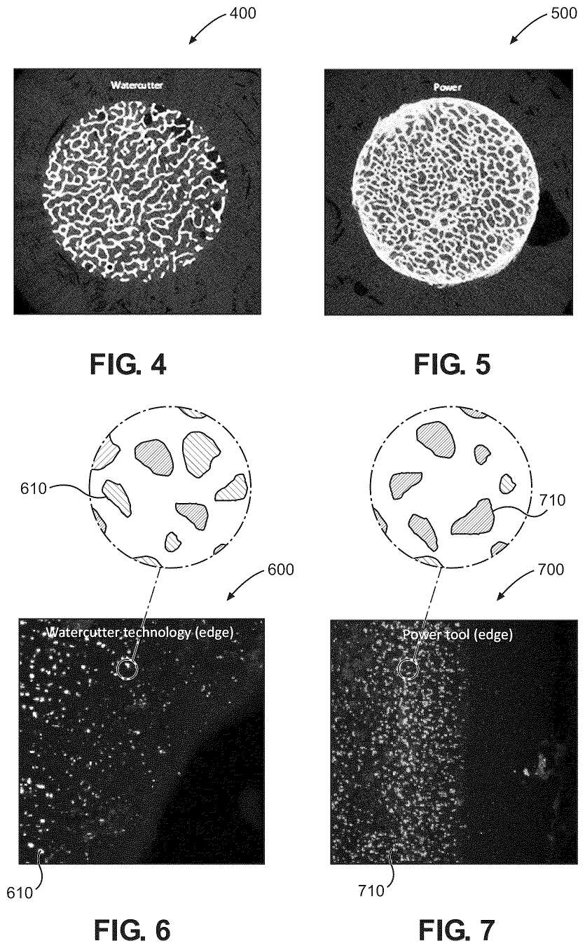

Embodiments of aspects of the present invention relate to providing a patient specific tissue graft by optimizing the shape, size, form, and architecture of the tissue for the patient anatomy and condition using a water cutter system. The method to produce patient specific tissue grafts is shown in FIG. 3 and includes, at 360, obtaining 2D or 3D images of the implant site and patient anatomy. 2D or 3D images, represented by data 302, could be obtained with the visualization system 300, using various imaging technologies including x-ray (radiographs), computed topography (CT), ultrasound, or magnetic resonance imaging (MRI). The desired shape, size, and architecture of the tissue is determined by the medical imaging scan. The scan is preferably used for computer-aided processing of the osteochondral tissue 200 by the water cutter system 10.

In one embodiment, a patient-specific osteochondral, chondral, or bone tissue graft is formed using a water cutter system. Computer systems and software programming tools are preferably used, at 370, to determine a 3D model of the tissue graft with exact shape, form, size, and architecture based on the dimensions and shape of the implant site or defect identified by the 3D images of the patient acquired from MRI. The water cutter system is preferably programmed, at 380, to form the exact size and shape of the desired graft. In another embodiment, patient specific structural bone tissue is generated using the water cutter system 10 using computer numerical control (CNC) manufacturing 320 and a robotic arm 330 to create 3D architectures. In one example, the structural bone graft is used as an implant for spinal interbody fusion. The exact patient-specific dimensions, shape, and architecture of the structural bone implant is preferably determined by the 3D image of the patient's spine, computer aided processing of the image, and adjusting for spinal alignment parameters, at 370. The CNC water cutter system with a robotic arm and 3D cutting capabilities can be used to generate a structural bone graft using the 3D model with the desired patient-specific size, shape, and modified architecture, at 380. Preferably forming the modified tissue architecture includes shaping or bending of a tissue graft in one or multiple axes to match a prescribe shape or curvature using controlled removal of slits, pores, or other prescribed regions of the tissue. An example of the method forming a patient-specific tissue graft shown in FIG. 3 is as follows. A 3D image of a patient's targeted implant site and surrounding anatomy is obtained. A boundary of the implant site is obtained and noted. A 3D model of a graft is created by utilizing the boundary of the implant site, a surface curvature, extrapolated from the patient's target implant site surrounding anatomy, and a depth, defined by a plane parallel to a plane normal to a point of curvature at the surface. A donor tissue is chosen that has a location with a close approximation to the 3D model of the graft. The donor tissue is visualized to determine a cutting path to shape or modify an architecture of the tissue. A cutting path is identified with imaging technology and computational methods. The graft is cut out of the donor tissue utilizing a fluid cutting process with computer numerical controls to cut the graft from the donor tissue along the cutting path.

### Fluid Cutting Parameters to Control Tissue Properties

The processing parameters of the fluid cutting system, at 370 and 380, directly impact the remaining surface of the cut tissue. Embodiments of the present invention impact the tissue micro-architecture, roughness, nanoroughness, density, surface energy, surface charge, as well as other parameters through modifications of the processing parameters. Examples of such parameters include fluid media, abrasive media, pressure, flow rate, nozzle diameter, cutting distance, cutting speed, dwell time, and processing time. The combination of parameters depends on the type of tissue being cut (e.g. bone, cartilage, skin, etc.) as well as the desired surface properties after cutting.

### Graft Recipient Implant Site Preparation

The recipient site needs to be prepared, at 385, to remove tissue such that it can be replaced with the graft 230, at 388. Preferably, shaping a tissue bed or removing autologous patient tissue in vivo during a surgical procedure, includes preparing a surgical site in a proper geometry removing tissues in a site-specific manner, to remove necrotic tissue, tumors, burns, or fibrous tissue; and removing foreign bodies or particles from native tissue, such as dirt, gravel, tattoo ink, projectiles, etc. In one example, the recipient site involves skin or dermal tissue and the fluid cutter system is used for debridement or dermabrasion.

Embodiments of aspects of the present invention utilize a water cutter system 10. The water cutting system minimizes tissue damage and improves native viability and matrix integrity of the tissue to provide an optimal healing environment with the implant. The water cutting system may or may not include abrasive media to support cutting. The additive may involve biocompatible and biodegradable materials which could include but not limited to salt crystals (e.g., sodium chloride, calcium sulfate, calcium phosphate, etc.), sugar crystals (sucrose, glucose, etc.), calcium carbonate, biodegradable polymers, tissue particle (e.g., bone or cartilage particles), ice or dry ice particles, and other biomaterials. In another embodiment, the water cutter system can use physiological buffered solutions such as phosphate buffered saline or solutions of controlled osmolarity (e.g., hyperosmotic solution) to maintain viability and biological properties of the tissue. In another embodiment, the hyperosmotic solution can be used to prevent dissolving of the additive crystals to enhance their cutting abilities.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A customized tissue graft for repair of a tissue defect, said tissue graft comprising:
a tissue matrix having tissue with a boundary surface, formed through a fluid cutting process, wherein the cutting process employs a fluid formed of a physiological buffered solution and the tissue maintains a native structure and/or viability at the boundary surface.

2. The tissue graft of claim 1, wherein the graft is in a cylindrical, oblong, rectangular, core, particulate, or irregular form.

3. The tissue graft of claim 1, wherein the tissue matrix is in particulate form with maintained tissue viability and/or native architecture particulate and is mixed with hydrogels, synthetic or natural materials, polymers, to form the tissue matrix.

4. The tissue graft of claim 1, wherein the tissue comprises soft and/or hard tissue sourced from a recipient or patient with the tissue defect or from donors.

5. The tissue graft of claim 4, wherein the tissue comprises musculoskeletal, neural, dermal, cardiovascular, ocular, nasal, costal, adipose, systems, or any tissue or organ type of a donor or autologous tissue.

6. The tissue graft of claim 1, wherein the tissue is osteochondral tissue with a cartilaginous layer and a bone portion, wherein a boundary of the graft maintains a native matrix and/or viability of the tissue through use of a fluid cutting process.

7. The tissue graft of claim 6, wherein the bone portion includes a rounded or angled surface edge to aide in implantation.

8. The tissue graft of claim 6, wherein the cartilaginous layer has a thickness of at least 0.050 mm and wherein the bone portion has a thickness of at least 0.010 mm.

9. The tissue graft of claim 1 wherein the cutting process further employs a particulate abrasive additive involving biocompatible materials.

10. The tissue graft of claim 6, wherein the bone portion contains additional tissue removal to allow shape manipulation.

11. The tissue graft of claim 1, wherein the tissue matrix includes conduits, pores, cuts, or a modified surface architecture or topology to improve integration.

12. A method of making a tissue graft comprising:
forming a modified surface, architecture, or shape of a donor tissue having a native matrix and/or viability to make the tissue graft, wherein the forming includes cutting the donor tissue with a fluid formed of a physiological buffered solution to maintain the native matrix and/or viability of the tissue at the modified surface.

13. The method of claim 12, wherein cutting the donor tissue includes performing defined cuts with a sample or water cutter system controlled with a CNC system.

14. The method of claim 12, wherein forming the modified tissue architecture while maintaining viability and native matrix includes visualizing and mapping the donor tissue and determining cutting paths using feedback from direct imaging and/or computational methods.

15. The method of claim 12, wherein forming the modified tissue architecture includes forming a patient specific tissue graft by matching the shape, size, form, and architecture of the tissue to a patient anatomy and condition based on 2D or 3D images of an implant site and the patient anatomy and condition.

16. The method of claim 12, wherein forming the modified tissue architecture includes:
forming pre-cut viable or non-viable chondral or osteochondral cores and complex, non-circular, or particulate osteochondral tissue shapes or
forming the architecture of the graft to create conduits, pores, or a modified surface architecture or topology to improve integration, with a bone or soft tissue graft.

17. The method of claim 12, wherein forming the modified tissue architecture includes cutting the donor tissue with a particulate abrasive additive involving biocompatible materials.

18. The method of claim 12, wherein forming the modified tissue architecture includes shaping or bending of a tissue graft in one or multiple axes to match a prescribe shape or curvature using controlled removal of slits, pores, or other prescribed regions of the tissue.

19. The method of claim 12 further comprising shaping a tissue bed or removing autologous patient tissue in vivo during a surgical procedure, wherein shaping further includes:
preparing a surgical site in a proper geometry;
removing tissues in a site-specific manner, to remove necrotic tissue, tumors, burns, or fibrous tissue; and
removing foreign bodies or particles from native tissue.

20. A method of forming a patient-specific tissue graft comprising:
obtaining a 3D image of a patient's targeted implant site and surrounding anatomy,
identifying and noting a boundary of the implant site,
creating a 3D model of a graft by utilizing the boundary of the implant site, a surface curvature, extrapolated from the patient's target implant site surrounding anatomy, and a depth, defined by a plane parallel to a plane normal to a point of curvature at the surface,
choosing donor tissue that has a location with a close approximation to the 3D model of the graft,
visualizing the donor tissue to determine a cutting path to shape or modify an architecture of the tissue,
identifying a cutting path with imaging technology and computational methods, and
cutting the graft out of the donor tissue utilizing a fluid cutting process that employs a fluid formed as a physiological buffered solution with computer numerical controls to cut the graft from the donor tissue along the cutting path.
